(19) United States
(12) Patent Application Publication
(10) Pub. No.: US 2026/0284261 A1
(43) Pub. Date: Sep. 24, 2026

(54) IMPLANT COMPOSITIONS AND METHODS FOR TENDON AND LIGAMENT REATTACHMENT

(71) Applicant: TETROUS, INC., Sherman Oaks, CA (US)

(72) Inventors: Andrew J. CARTER, June Lake, CA (US); Ian McRURY, Medway, MA (US); Bradley E. PATT, Sherman Oaks, CA (US); Ivan POPOFF, Coogee (AU); Doron SHER, Bondi Junction (AU); Patrick MAY, St. Ives (AU)

(21) Appl. No.: 19/569,937

(22) Filed: Mar. 17, 2026

Related U.S. Application Data
(60) Provisional application No. 63/773,980, filed on Mar. 18, 2025.
(60) Provisional application No. 63/910,765, filed on Nov. 3, 2025.

(51) Int. Cl. A61L 27/36

(52) U.S. Cl. CPC A61L 27/3608; A61L 2430/02; A61L 2430/10

(57) ABSTRACT

An implant system for augmenting reattachment of a tendon or ligament graft to a bone, the implant system, comprising the tendon or ligament graft, an optional fixation device attached to an end of the tendon or ligament graft, and a fiber implant (cannulated or non-cannulated), comprising a plurality of cut fibers (e.g., demineralized bone fibers) molded into a shape of a tubular prism, a cylinder, a truncated cone, a rectangular prism with rounded ends, a tapered rectangular prism with rounded ends, a cylindrical segment, a multi-edged rod (e.g., hexagonal rod), or a strip, where the tendon or ligament graft is inserted with the fiber implant in a tunnel or socket of the bone.

### CROSS-REFERENCE TO RELATED APPLICATION(S)

This application claims priority to and the benefit of U.S. Provisional Application No. 63/910,765, filed Nov. 3, 2025, and U.S. Provisional Application No. 63/773,980, filed Mar. 18, 2025, the entire contents of both of which are herein incorporated by reference.

### FIELD OF THE INVENTION

The field of the invention relates to composition and methods of bone fiber implants made from cortical bone in which a plurality of demineralized bone fibers forms a conical or strip shape to augment tendon or ligament reattachment in a subject.

### BACKGROUND

The background description includes information that may be useful in understanding the present disclosure. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

When tendon or ligament tissues or grafts are placed either in apposition to bone, as in the case of rotator cuff repair or in bone tunnels as in anterior cruciate ligament (acl) repair, the tendon-bone enthesis is not recreated and scar tissue forms instead. Scar tissue does not allow efficient transfer of loads between the tendon or ligament and bone and is thus prone to re-tear or failure. This is a problem and concern to orthopedists.

### SUMMARY OF INVENTION

The inventors of the presently disclosed subject matter have discovered an advantageous implant device for tendon and ligament reattachment to bone. The presently disclosed implants are capable of: 1) providing an osteoinductive and osteoconductive implant to facilitate regeneration of the enthesis, 2) augmenting the effectiveness of the fixation device, and 3) self-stabilizing during surgery unlike other implants. Accordingly, aspects of embodiments of the present invention are directed to a means of improving the fixation of implants and tissue to bone through the use of an implant, which may, for example, be composed of fibers of demineralized bone, collagen fibers, polymer fibers, and/or resorbable polymer fibers, and formed into an appropriate shape. In particular, the fiber implant made of a plurality of fibers has a shape selected from a tubular prism with at least one protuberance, a rectangular prism with rounded ends, a tapered rectangular prism with rounded ends, a cylindrical segment, a tubular shape, a conical (truncated cone) shape, a strip, and a multi-edge rod shape.

In some embodiments, the fiber implant is cannulated and may be posited using a guide wire.

In some embodiments, the fiber implant is a cannulated tubular prism with tapered ends and a and at least one protuberance. The cannulated tubular prism may have a width of about 3 to about 8 mm, a height of about 3 to about 10 mm, and a length of about 10 to about 100 mm, about 10 to about 80 mm, about 10 to about 70 mm, about 10 to about 60 mm, about 10 to about 50 mm, about 20 to about 80 mm, about 20 to about 70 mm, about 20 to about 60 mm, or about 20 to about 50 mm. In some embodiments, the at least one protuberance extends about 1 to 5 mm outward from the implant.

In some embodiments, a method of augmenting reattachment of a tendon or ligament graft to a first bone of a subject in need thereof, comprises using the fiber implant as disclosed herein.

In additional embodiments, a method of augmenting reattachment of a tendon or ligament graft to a first bone of a subject in need thereof, comprises positing the cannulated fiber implant as disclosed herein in contact with the tendon or ligament graft in a tunnel or socket of the first bone of the subject.

In some embodiments, a method of augmenting reattachment of a tendon or ligament graft to a bone of a subject in need thereof, comprises positing the cannulated fiber implant as disclosed herein in contact with the tendon or ligament graft in a tunnel or socket of the bone of the subject, wherein the tunnel or socket, the method comprising positing the cannulated fiber implant into the tunnel or socket to secure the graft to the bone. The cannulated fiber implant may be placed using a guide wire and/or by threading through attached sutures. In some exemplary embodiments, the cannulated fiber implant is the cannulated rectangular prism having at least one protuberance as disclosed herein.

In exemplary embodiments, the bone is selected from the tibia, the femur, the humerus, the ulna, or the radius. In other exemplary embodiments, the method comprises any of the methods above, wherein the tendon or ligament graft is for anterior cruciate ligament (ACL) repair and comprises placement of the implants in opposing bones, e.g., the femur and the tibia.

In specific embodiments, the method of augmenting reattachment of a tendon or ligament graft to a first bone of a subject in need thereof, comprises any of the methods above, wherein the cannulated fiber implant is the cannulated rectangular prism having at least two protuberances and wherein the plurality of cut fibers are DBF.

In some embodiments of the present invention, the implant is placed at the interface between the tendon and the bone. The implant according to some embodiments of the present invention serves to improve the integration between the tendon and bone and facilitate recreation of the enthesis.

In an embodiment, the implant device for tenodesis or anterior cruciate ligament (acl) reconstruction is tubular or shaped like a bag, having a diameter of about 3 to about 12 millimeters (mm), a length of about 5 to about 40 mm. In a preferred embodiment, the tubular or bag-like implant device has a diameter of about 6 mm. In other embodiments, the cylindrical implant device has a diameter of 3, 4, 5, 6, 7, 8, 9, 10, 11, or 12 mm and a length of 10, 11, 12, 13, 14, 15, 16, 17, 18, 19, 20, 25, 30, 35, or 40 mm.

In some embodiments, the tubular implant has at least one slit along part of the length of the implant to allow the implant to deform and therefore, accommodate a graft placed inside the tubular implant. In some embodiments, the tubular implant comprises a hole through which sutures and/or tendon or ligament graft are threaded.

In an embodiment, the implant device for tenodesis or acl reconstruction is conical (e.g., a truncated cone), having a diameter of about 3 to about 12 millimeters (mm) and a length of about 3 to about 15 mm. In a preferred embodiment, the conical implant device has a diameter of about 6 mm, tapered to about 5 mm at its distal end. In other embodiments, the conical implant device has a diameter of 3, 4, 5, 6, 7, 8, 9, 10, 11, or 12 mm, with a taper towards the distal end of 1, 2, or 3 mm and a length of 3, 4, 5, 6, 7, 8, 9, 10, 11, 12, 13, 14, or 15 mm.

In some embodiments, the conical implant device has a central hole so that sutures can be threaded through the implant. The cylindrical implant device may have a hole forming an inner diameter of 1, 2, 3, 4, 5, or 6 mm.

In some embodiments, the conical implant device has a slit along the entirety of the implant length to allow for sutures to be threaded through the implant after it has been placed (e.g., in a bone tunnel or bone cavity). The width of the slit may be about 1 to about 5 mm.

In an embodiment, the implant for tenodesis or acl reconstruction is a strip having a width of about 3 to about 8 mm, a thickness of about 0.5 to about 4 mm, and a length of about 10 to 60 mm. In some embodiments, the strip implant device has a width of 3, 4, 5, 6, 7, or 8 mm; a thickness of 0.5, 1, 2, 3, or 4 mm; and a length of 10, 15, 20, 25, 30, 35, 40, 45, 50, 55, or 60 mm.

Typical embodiments of the present invention include an implant for tendon-to-bone reattachment, the implant is made of demineralized bone. In some embodiments, the implants are made of a plurality of fibers cut from demineralized bone, the plurality of fibers forming a cylinder, truncated cone, or a strip. In particular, the tubular implant may be used in biceps tenodesis where the tubular implant is placed in a bone tunnel together with threaded sutures with suture anchors for fixation. In particular, the strip implant may be used in biceps tenodesis where the strip is placed in a bone cavity against the bone, followed by a tendon graft, which is then fixed into place with an interference screw.

The implant may be made of demineralized bone fibers (DBF), biocompatible polymer fibers, collagen fibers, and/or resorbable polymer fibers, and the implant may be provided with additional fibers of the same or different type as disclosed herein. The implant made of the DBF fibers is both osteoinductive and osteoconductive.

The inventive subject matter also includes methods of fabricating the fiber implant. In some embodiments, the method of fabricating the presently disclosed fiber implant includes dispersing a plurality of fibers (DBF, biocompatible polymer fibers, collagen fibers, and/or resorbable polymer fibers) in a fluid, wherein the weight of the fibers and the volume of fluid are in a ratio of between about 1 gram of fibers to about 3 to about 60 mls of fluid, followed by providing the fluid-dispersed plurality of fibers with suitable pressure into a vented mold to thereby drain the fluid out of the mold, allowing the fibers to fill the mold and assume its shape.

In some embodiments, suitable pressure is necessary to cause the fiber fluid slurry to flow and to enter the mold and the fluid to be vented from the mold. Suitable pressure ensures the fibers are not compressed in the mold and retains porosity in the molded fiber structure.

In exemplary embodiments, the ratio of weight of fibers (e.g., DBF) to fluid volume range from 1 gram fibers to 60 mls fluid, 1 gram fibers to 55 mls fluid, 1 gram fibers to 50 mls fluid, 1 gram fibers to 45 mls fluid, 1 gram fibers to 40 mls fluid, 1 gram fibers to 35 mls fluid, 1 gram fibers to 30 mls fluid, 1 gram fibers to 25 mls fluid, 1 gram fibers to 20 mls fluid, 1 gram fibers to 15 mls fluid, 1 gram fibers to 10 mls fluid, 1 gram fibers to Preferably, the fibers and the fluid are in a ratio of 1 gram of fibers in about 3 mls to 20 mls of fluid. More preferably, the fibers and the fluid are in a ratio of 1 gram of fibers in about 3 mls to 10 mls of fluid.

In exemplary embodiments the fluid used is water. Other suitable fluids include saline and phosphate buffered saline.

In additional embodiments, the method disclosed above and herein also includes heating the plurality of fibers in a mold. Preferably, the heating occurs at or between about 35 to 55 degrees Celsius (35 to 55° C.). In some embodiments, the method also includes lyophilizing the plurality of fibers. In some embodiments, the method also includes vacuum drying the plurality of fibers.

The inventive subject matter also includes a method for making the presently disclosed implant. In typical embodiments, the method for making the presently disclosed implant includes dispersing a plurality of fibers suspended in fluid with pressure (under pressure) into a vented mold thereby draining the fluid through the mold. The method may further include heating the plurality of fibers in the mold. Heating of the plurality of fibers may occur at or between 35 to 55° C. or 45 to 55° C. In example embodiments, the fibers (e.g., demineralized bone fibers) are subsequently lyophilized.

Additional embodiments of the contemplated subject matter include a method of facilitating the surgical placement of an orthopedic bone implant wherein the method includes augmenting a tendon graft with cylindrical or strip implant as disclosed herein.

### BRIEF DESCRIPTION OF THE DRAWINGS

FIG. 1 shows a cross-section view of the apparatus for water assisted injection molding of DBF that provides for manufacture of a cannulated implant using the water assisted injection molding process. The DBF fibers 1 are loaded into a syringe 2, the distal end of the syringe is fitted into an adapter 3, attached to which is a detachable mold 4, where the mold is tapered towards its distal end and has vents 5 along its length, and a vented end 6 that also serves to hold a guide wire 7 to form the cannulation.

FIG. 2 shows a truncated cone implant 8, with a center hole 9.

FIG. 3 shows a truncated cone implant 10, with center hole 9 and a slit 11.

FIG. 4 depicts graft fixation (e.g., acl) using a truncated cone implant device 8 placed against the bone in femoral socket 12 in femur 40 and threaded with sutures 13a, 13b which are also attached to, or looped around, the graft 14 through hole 9 with a fixation device 15 (e.g., a button fixation device) securing the superior end of the graft 14. The distal tunnel 16 allows the fixation device to be passed and positioned against the outer cortex of the femur 40.

FIG. 5A shows a rectangular strip shaped implant 17.

FIG. 5B shows a rectangular strip shaped implant 18, with a centrally placed groove 19 that serves to allow the implant to fold.

FIG. 5C shows a rectangular strip shaped implant 20, with a semicircular cross-section 21.

FIG. 5D shows a bow-tie shaped implant 22 with a centrally placed groove 19 to facilitate folding of the implant.

FIG. 5E shows a bow-tie shaped implant 23 with a centrally placed groove 19 to facilitate folding and a semicircular cross-section 24.

FIGS. 6A-6D depict methods where a tendon 25 is whipstitched to a suture 28 that is incorporated into an introducer 26. An implant 18 is placed in a pre-drilled socket 45 in the bone in FIG. 6A. In FIG. 6B, the introducer 26 is placed in the socket 45, and in so doing the tendon 25 is placed in contact with and surrounded by the implant 18. The interference screw 27 is inserted into the socket 45 compressing the tendon 25 against the implant 18 as shown in FIG. 6C. In FIG. 6D a variant of the procedure is shown where the implant 17 is a strip that is placed on one side of the socket 45.

FIG. 7A depicts an acl replacement surgery in which fixation in the femur 40 with graft 14 is effected with implant 17 placed in the femoral socket 12 with graft 14 secured with suture(s) 13a (inside bone), 13b (outside bone) extending through a hole at the distal end (opposite the opening) of the femoral socket 12 through the femoral tunnel 16 to the surface of the femur 40 for placement of a fixation device 15 (e.g., a button fixation device) thereon, with opposing fixation in the tibia 60 by securing graft 14 using an interference screw 41, with another (second) implant 17 placed in the tibial tunnel 16′, as indicated.

FIG. 7B depicts an acl replacement surgery in which fixation in the femur 40 with graft 14 is effected with implant 17 placed in the femoral tunnel 16 with graft 14 secured with suture(s) 13a, 13b extending through the femoral tunnel 16 to the surface of the femur 40 for placement of a fixation device 15 (e.g., a button fixation device) thereon, with opposing fixation in a tibial socket 45′ by securing graft 14 using a cross-pin (or cross-screw) 54, with another (second) implant 17 placed in the tibial socket 45′, as indicated.

FIG. 7C depicts an acl replacement surgery in which fixation in the femur 40 with graft 14 is effected with implant 17 placed in the femoral socket 45 with graft 14 secured with suture(s) 13a extending through the femoral socket 45 to the cross-pin 54 fixation, with opposing fixation in a tibial socket 45′ by securing graft 14 using another (second) cross-pin 54, with another (second) implant 17 placed in the tibial socket 45′, as indicated.

FIG. 8 depicts a mold for making strip-like implants. The mold has a lid 29, a body 30 and both have holes 31 for drainage.

FIG. 9 depicts an introducer 33 for use with conical and cylindrical implants (8, 10) with a feature designed to have a rod with a diameter corresponding to the diameter of the implant cannulation 32. The figure also shows an implant 8 positioned on the introducer 33.

FIG. 10A depicts an implant 34 with cannulation 35 where the implant is a rectangular prism with rounded ends.

FIG. 10B depicts an implant 36 with cannulation 35 where the implant is a tapered rectangular prism with rounded ends.

FIG. 10C depicts an implant 37 with cannulation 35 where the implant is a cylindrical segment.

FIGS. 11A-11C depict the steps in implantation of any of the implants of FIGS. 10A-10C. FIG. 11A shows a cross-sectional view of a bone (tunnel or socket) 39 where the 4 strands of a graft 38 (e.g., acl graft) have been introduced. FIG. 11B shows placement of a guide wire 55 along the axis of the (tunnel or socket) 39. FIG. 11C shows an implant 34 that has been introduced into the (tunnel or socket) 39 using the guide wire 55 to guide the placement.

FIG. 12A depicts a hexagonal implant 42 designed to fit into the driver hole of a fenestrated interference screw 41 to be used for fixation of an acl graft 38 in the bone (tunnel or socket) 39.

FIG. 12B depicts fixation of a graft 38 (e.g., acl) in a bone (tunnel or socket) 39 using a fenestrated interference screw 41 with a hexagonal implant 42 designed to fit into the driver hole of the screw.

FIGS. 13A-13B depict two different perspectives of a tubular or bag-like implant 43 with a rounded end that has a hole 44 to allow passage of the suture in an acl graft construct.

FIGS. 14A-14B depict a tubular or bag-like implant 43 with a rounded end that has a hole 44 to allow passage of the suture in an acl graft construct, and has one slit 46 (FIG. 14A) or two slits 46 (FIG. 14B) along part of the implant length.

FIG. 15 depicts a cross-section view of a tubular or bag-like implant 43 placed in contact with the bone in femur socket 12 in a femur 40 for fixation of ligament or tendon graft 14 with sutures 13a, 13b attached to the tendon graft 14 and the suture(s) 13a, 13b extending through hole 44 in the implant and a hole at the distal end (opposite the opening) of the femoral socket 12 through the femoral tunnel 16 to the surface of the femur 40 for placement of a fixation device 15 (e.g., a button fixation device) thereon.

FIGS. 16A-16E depict exemplary devices used for forming the tubular implants (43) of FIGS. 13A, 13B, 14A, and 14B. FIGS. 16A and 16B depict a sheet mold having a body 30 (FIG. 16A), a lid 29 (FIG. 16B), and both the lid 29 and body 30 have holes for drainage 31. FIG. 16C depicts a mandrel 47 with a region 48 that defines the internal diameter of the cylinder and a smaller diameter region 49 that defines the cannulation hole in the implant. The outer diameter of the implant is defined by the outer wall of the mold 50 (FIG. 16D) which has two identical halves held together by a clamp ring (not shown). FIG. 16E shows the fiber sheet 43 wrapped around the mandrel 47 placed in one of the molds 50 prior to the second mold half being placed.

FIG. 17 is a schematic of an implant 51 having a tapered tubular prism shape with cannulation 35 and three protuberances 52, as indicated.

FIGS. 18A-18B show a photograph and a schematic of implant 53, respectively, the implant made of DBF with cannulation 35, having a tapered tubular prism shape with rounded ends and two protuberances 52, as indicated.

### DETAILED DESCRIPTION

Aspects of the embodiments of the present invention are directed to an approach for augmenting bone repair and healing using demineralized bone fiber (DBF), collagen fiber, polymer fiber, and/or resorbable polymer fiber implants.

In other aspects, embodiments of the present invention include DBF, collagen, polymer, and/or resorbable polymer fiber implants, and methods of forming DBF, collagen, polymer, and/or resorbable polymer fibers implants, and kits providing formed DBF, collagen, polymer, and/or resorbable polymer fiber implants for use as an interface between the bone and the ligament or tendon to be repaired.

For example, a fiber implant as disclosed herein, may be used in the bone tunnels of a soft tissue ligament replacement such as an acl (anterior cruciate ligament) surgery where a hamstring or tendon autograft is fixed into a bone tunnel.

As used herein “implant,” “fiber implant,” “implant of the present disclosure,” and like terms are used interchangeably to refer to a suitably shaped fibrous implant made using demineralized bone, demineralized bone fibers (DBF), collagen fibers, polymer fibers, and/or resorbable polymer fibers. Exemplary implants are denoted herein 8, 10, 17, 18, 20, 22, 23, 34, 36, 37, 42, 43, 51, 53. Examples of DBF, collagen, polymer, and/or resorbable polymer fibers as disclosed herein and also disclosed in U.S. Pat. Nos. 9,486,557, 9,572,912, WO 2016/123583, and US 2020/0397586, the entire contents of all of which are incorporated herein by reference. As disclosed herein, non-limiting examples of a suitably shaped (e.g., by molding) implant include: truncated cones (8, 10), a sheet or strip (17, 18, 19, 20, 22, 23), a rectangular prism (34, 36) , a cylindrical segment (37), a hexagonal rod (42), tubular (e.g., hollow tube or bag) (43), a tubular prism (51, 53) with rounded ends and at least one protuberance 52 along its length.

As used herein, “providing,” “placing,” and “positing” are used interchangeably to refer to the action of placing the relevant object (e.g., the fiber implant, the fixation device, the tendon or ligament graft, or the guide wire) in the proximal bone or bones (e.g., a tunnel and/or a socket of the bone(s)) for repair of the relevant tendon or ligament.

As used herein, “graft” refers to the one or more strands to replace and serve the function of the subject's previous graft in need of reattachment or to replace the subject's native tendon or ligament in need of repair.

As used herein, a “tunnel” in the bone for attachment of the tendon or ligament refers to a drilled passage in the bone(s) having an opening at each end, through which the tendon or ligament graft are extended through. For example, the femoral tunnel 16 extends from femoral socket 12 as depicted in FIGS. 4, 7A, and 15, with the opposing tibial tunnel 16′.

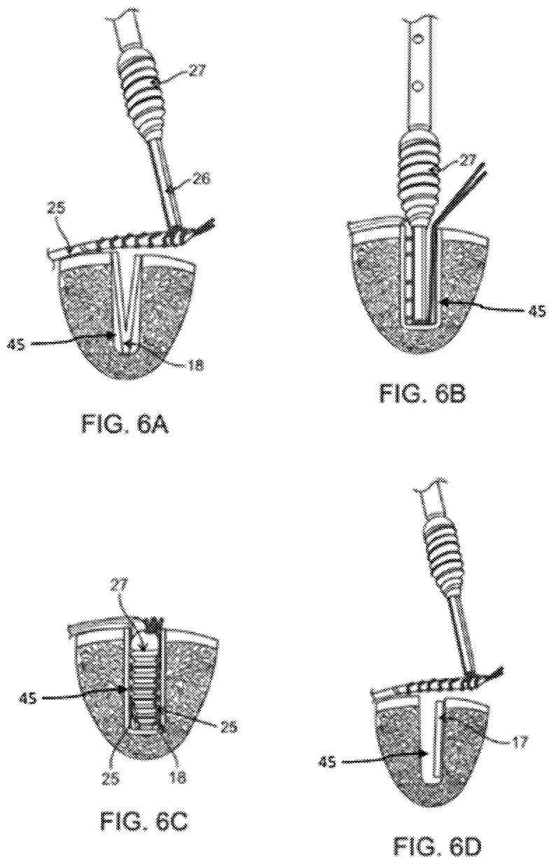

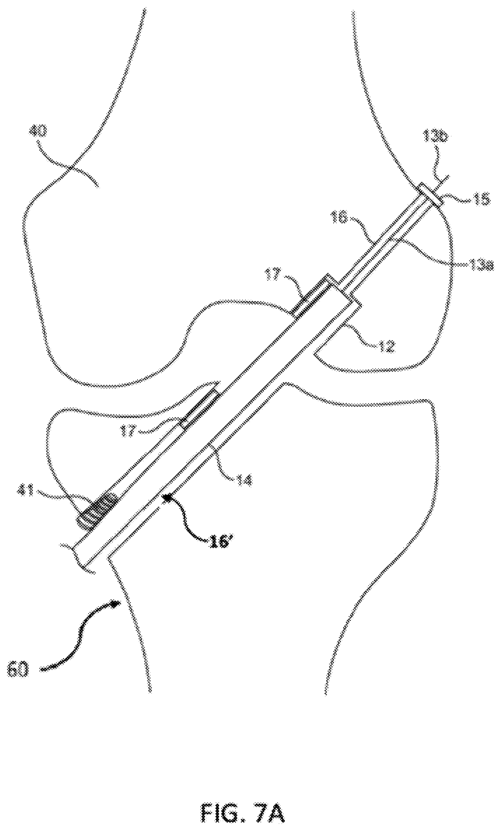

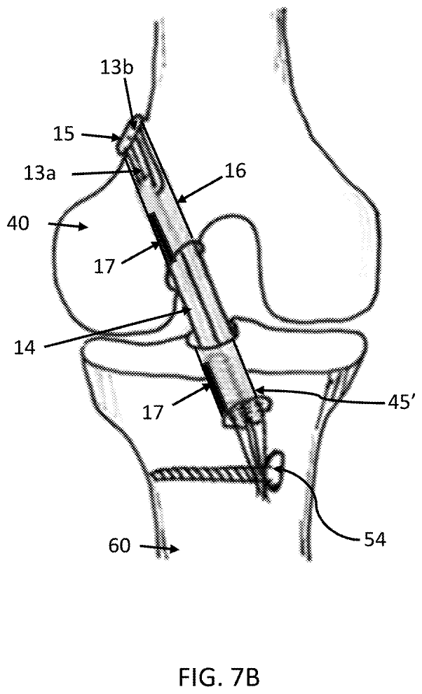

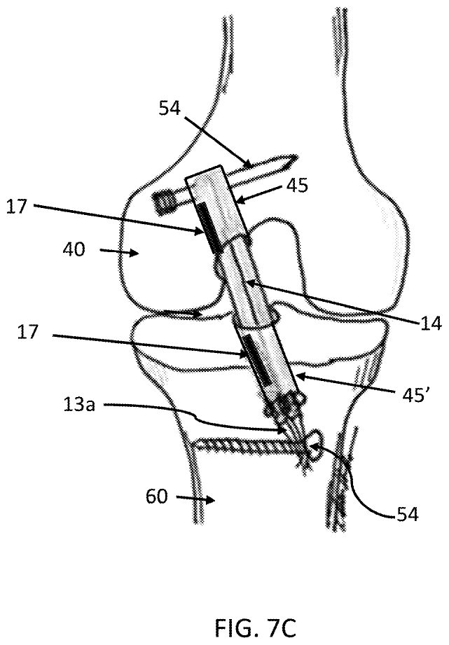

As used here in, a “socket” made in a bone refers to a cavity space drilled or carved in the bone in which any implant of the present disclosure may be placed. The socket has a proximal end and a distal end and at least one of the ends is open. In some embodiments, a socket has a closed distal end and an open proximal end as depicted by socket 45, 45′ in FIGS. 6A-6D, 7B and 7C, where the proximal end is at a surface of the bone. In other embodiments, a socket 12, 12′ as depicted in FIGS. 4, 7A, and 15 also refers to a cavity space in a bone having a hole at its distal end, through which sutures 13a, 13b may be passed through to extend into the femoral tunnel 16. Typically, the proximal opening of the socket 12, 12′ has a larger width or diameter than the diameter or width of the hole at the distal end.

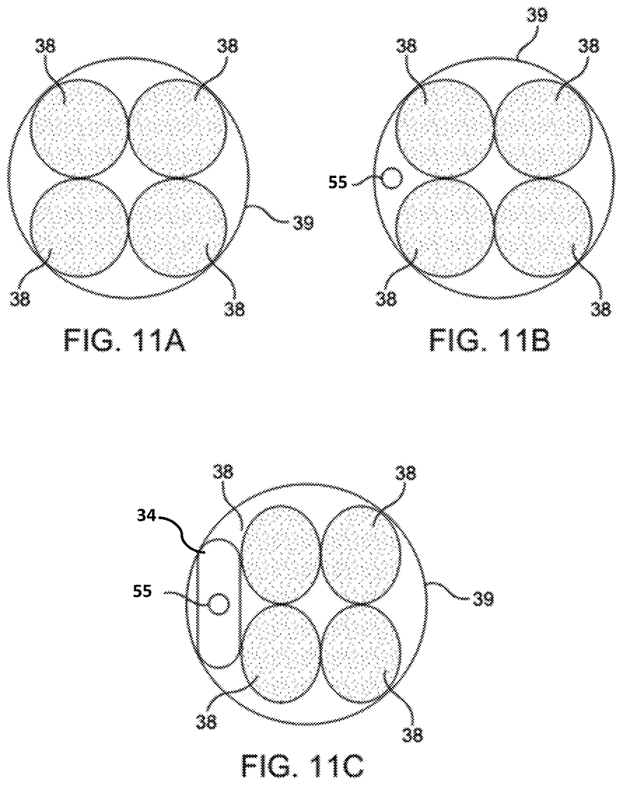

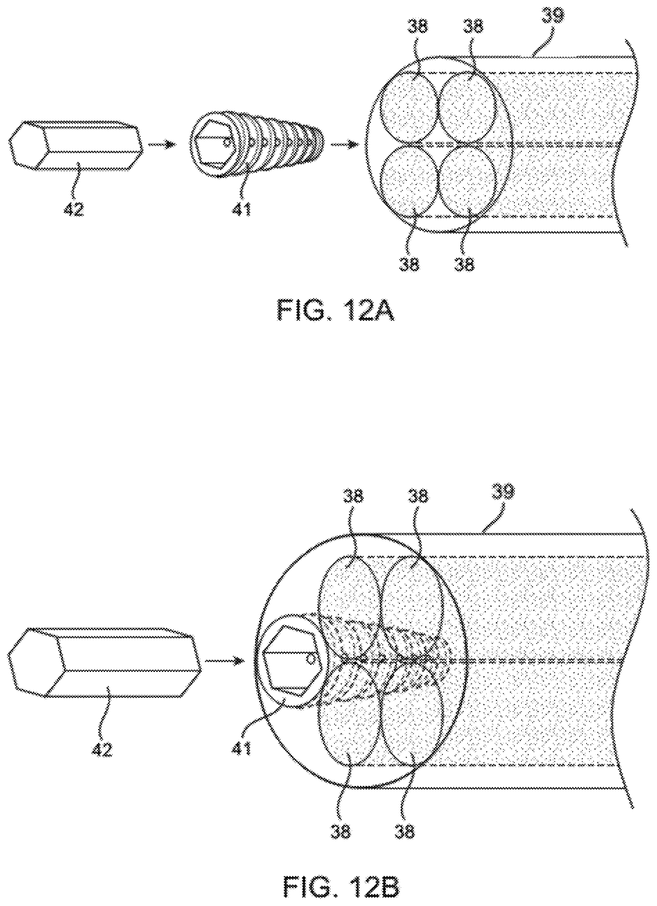

With reference to the (tunnel or socket) 39 in FIGS. 11A, 11B, 11C, 12A, and 12B, it is noted that the formation of a tunnel or a socket in the bone for the graft and any of the implants disclosed herein will be determined by the skilled person (e.g., surgeon). Accordingly, the (tunnel or socket) represents the possibility of either to be used with the disclosed implant. Furthermore, the use of a tunnel 16 or socket 12, 45 as disclosed in examples herein, may be modified for use with the other, such that implants disclosed using a tunnel are suitable for use with a socket with the corresponding modifications for fixation, and vice versa.

As used herein, a “hole” 9 or 44 in fiber implants 8 and 43, respectively, is an opening in the implant through which a suture or sutures and/or a graft may be threaded or passed through.

Additionally, collagen, polymer, and/or resorbable polymer fiber implants are made following the methods disclosed herein for DBF with a substitution or addition of the collagen, polymer, and/or resorbable polymer fibers. While DBF fibers are exemplified herein, these other fiber forms may also be used to form the presently disclosed fiber implants.

Resorbable polymers are biocompatible polymers capable of resorbing in the body and have a physical strength to form a fiber or particle at room temperature. Non-limiting examples of resorbable polymers include silk, collagen (including Types I to V and mixtures thereof), and proteins comprising one or more of the following amino acids: alanine, arginine, asparagine, aspartic acid, cysteine, glutamine, glutamic acid, glycine, histidine, isoleucine, lysine, methionine, phenylalanine, proline, serine, threonine, tryptophan, tyrosine and valine; polysaccharides, including alginate, amylose, carboxymethylcellulose, cellulose, chitin, chitosan, cyclodextrin, dextran, dextrin, gelatin, gellan, glucan, hemicellulose, hyaluronic acid, derivatized hyaluronic acid, oxidized cellulose, pectin, pullulan, sepharose, xanthan and xylan; resorbable polyesters, including resorbable polyesters made from hydroxy acids (including resorbable polyesters like poly(lactides), poly(glycolides), poly(lactide-co-glycolides), poly(lactic acid), poly(glycolic acid), poly(lactic acid-co-glycolic acid), poly(dioxanones), polycaprolactones and polyesters with one or more of the following monomeric units: glycolic, lactic; trimethylene carbonate, p-dioxanone, or ε-caprolactone, and resorbable polyesters made from diols and diacids; polycarbonates; tyrosine polycarbonates; polyamides (including synthetic and natural polyamides, polypeptides, and poly(amino acids)); polyesteramides; poly(alkylene alkylates); polyethers (such as polyethylene glycol, PEG, and polyethylene oxide, PEO); polyvinyl pyrrolidones or PVP; polyurethanes; polyetheresters; polyacetals; polycyanoacrylates; poly(oxyethylene)/poly(oxypropylene) copolymers; polyacetals, polyketals; polyphosphates; (phosphorous-containing) polymers; polyphosphoesters; polyalkylene oxalates; polyalkylene succinates; poly(maleic acids); biocompatible copolymers (including block copolymers or random copolymers); and hydrophilic or water soluble polymers, such as polyethylene glycol, (PEG) or polyvinyl pyrrolidone (PVP), with blocks of other biocompatible or biodegradable polymers, for example, poly(lactide), poly(lactide-co-glycolide), or polycaprolactone or combinations thereof. Resorbable polymers also include cross-linked polymers, and include, for example, cross-linked collagen, as well as functionalized polymers. In some embodiments, resorbable polymers are resorbable polyesters.

The popularity of demineralized bone matrix (DBM)-based products is based on the ability to induce bone formation through expression of inherent non-collagenous proteins that stimulate some cell types present at the graft site to differentiate into bone forming cells. This induction of bone formation process is referred to as “osteoinduction” and is due to the natural presence of bone morphogenic proteins (BMPs). DBM also provides a scaffold for these cells to populate and spread throughout in a process known as “osteoconduction.” Demineralized bone in the form of a fiber, known as Demineralized Bone Fiber (DBF) has a physical form that has been shown to optimize and enhance the osteoconductive performance of DBM. In some embodiments of the present invention, a composition and method of manufacture of DBF fibers is as disclosed in U.S. Pat. Nos. 9,486,557 and 9,572,912, supra. When DBM or DBF is combined with osteogenic cells that are capable of forming bone, the three mechanisms of bone healing (e.g., osteoinduction, osteoconduction, and osteogenesis) are combined.

In exemplary embodiments, the DBF implant is dried so that the implant has sufficient rigidity to allow it to be pushed into a pre-formed hole or tunnel. The DBF fibers may be easily formed into any of the required implant shapes using molding or wet laying processes prior to drying. Optionally a heating step may be utilized which has been shown to impart even greater cohesion to formed DBF implants without affecting the implant's osteoinductivity.

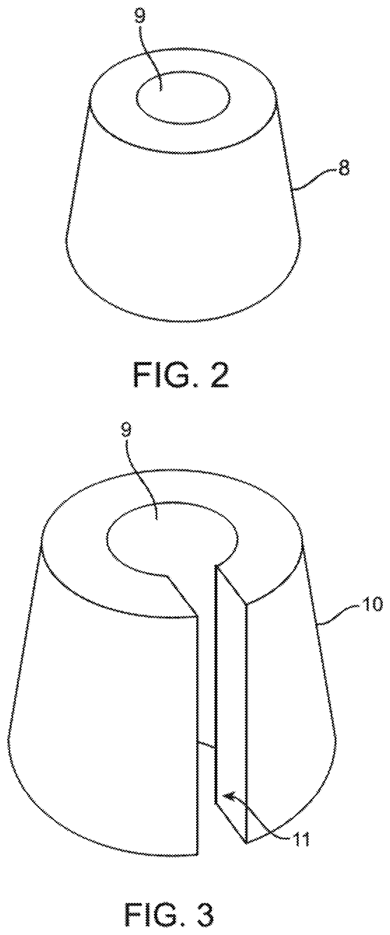

Accordingly, the inventors of the presently disclosed subject matter have discovered an advantageous fibrous implant 8 or 10 that is at once capable of: 1) stimulating reformation of the enthesis 2) augmenting the effectiveness of the fixation such as with an interference screw, and 3) self-stabilizing during surgery unlike other implants. Accordingly, aspects of embodiments of the present invention are directed to a means of improving the fixation of implants and tissue to bone through the use of fibrous implant 8 or 10, which may, for example, be composed of fibers of demineralized bone and formed into a conical shape as shown in FIGS. 2 and 3.

FIG. 4 shows the placement of an implant 8 for anterior cruciate ligament (acl) replacement surgery using a fixation device such as a suspensory fixation device, e.g., Smith & Nephew's Endobutton® or Ultrabutton®. The surgical technique is not changed from the usual manner with a socket 12 (or tunnel) being drilled from the desired insertion site within the joint at the desired trajectory to receive graft 14. The graft 14 (e.g., an autologous hamstring) is attached to the suspensory fixation device 15 using suture 13a, 13b, where the suture tail 13b is attached to the suspensory fixation device 15 that is passed through the center hole 9 of the truncated cone (i.e., conical) implant 8. Suture 13a refers to the suture inside the bone. As set forth in FIG. 4, the suture 13a, 13b is threaded through the hole 9 of implant 8 in socket 12 and up through the femoral tunnel 16 and the suspensory fixation device 15 is flipped in the normal manner according to conventional procedures. As exemplified in FIG. 4, the implant 8 is positioned at the top of the femoral socket 12, sandwiched between the tendon and the bone where the implant 8 when made from DBFs having BMPs, has the desired effect of stimulating an improved enthesis regeneration.

It will be recognized by those skilled in the art that there are several types of suspensory fixation device (e.g., Endobutton^{®}) available to the skilled person and known in the art, and each type of fixation device has a corresponding surgical technique and method of placement. However, the implant device (e.g., implant 8 or 10) of the present invention is used and placed as disclosed herein using all device types. Where the example shows femoral fixation in the knee the suspensory fixation technique can be applied to other anatomies including, for example, the tibial knee, humerus, and elbow.

In a preferred embodiment, the implant device 8 is dried or lyophilized. In this form the device is stiff and insertion into the cavity is easiest.

The truncated cone device 8 is designed to be compatible with the suture and button device with which it is being used. As such the diameter of the center hole 9 may vary from 0.5 mm to 6 mm, while the length of the implant 8 may vary from 3 to 15 mm. Similarly, the diameter of the implant 8 corresponds to the diameter of cavity required for the graft and may vary from 3 to 15 mm. The optional taper along the length of the device is designed to aid insertion. Accordingly, in some embodiments, the truncated cone device has more than one diameter as a tapered implant has a diameter at one end that is larger than the smaller diameter at the tapered end.

The slotted truncated cone device 10 shown in FIG. 3 may be used in a similar way to the truncated cone device 8 in FIG. 2, with the exception that the slot 11 allows the implant device 10 to be placed on the suture in the desired location without the need to thread the button of the suspensory fixation device 15 through the slotted implant device 10. Accordingly, the slotted implant 10 may be provided to a graft implant placed in the bone in a previous surgical step.

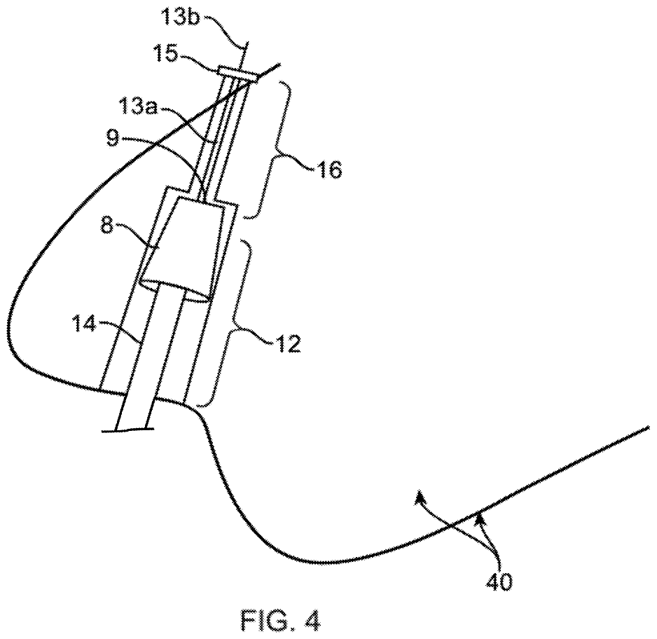

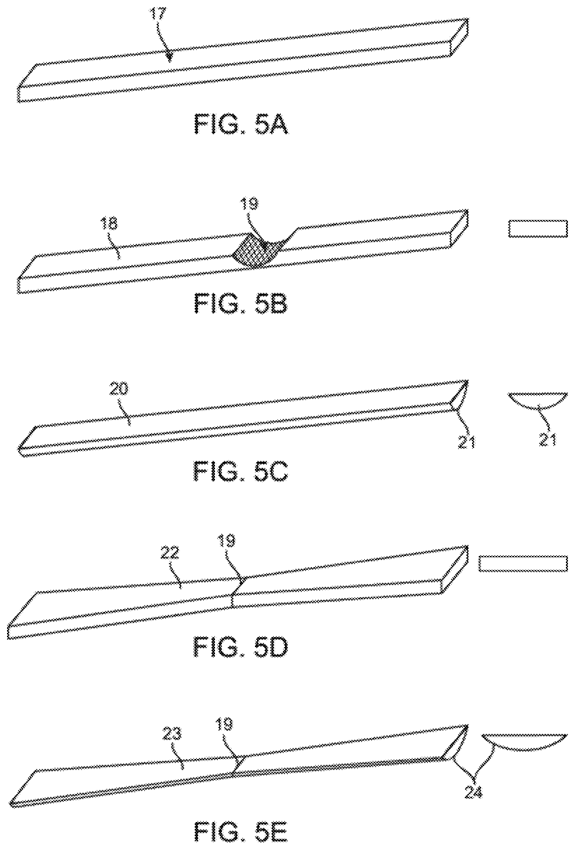

In some instances, it is desired to place the implant along the axis of the ligament or tendon rather than at the distal end. To facilitate this use, various implant configurations are disclosed herein that are fabricated from demineralized bone either by machining cortical bone before or after it has been demineralized or from molding fibers (e.g., DBFs or polymer fibers). Demineralized bone strip implants 17, 18, 20, 22, or 23 are shown in FIGS. 5A-5E. FIG. 5A shows a simple rectangular prism or cuboid shaped implant 17. FIG. 5B shows a variant of this where the implant 18 has a centralized feature 19 that provides a point where the implant can be folded. FIG. 5C. shows a variant where the implant 20 has a semi-circular profile. FIG. 5D shows a bow-tie shaped implant 22 that has a central feature 19 to provide ease of folding. FIG. 5E shows a variant of the bow-tie shaped implant 23 with a semi-circular cross section 24.

There are a number of surgical techniques that can be used to place and fix a tendon or ligament into a bone tunnel or socket and the implants of the examples in FIGS. 5A-5E may be used to improve enthesis healing. FIGS. 6A-6C depict a surgical technique where a tendon 25 is stitched (e.g., whipstitched) to an introducer 26 using suture 28. An implant 18 is placed in a pre-drilled hole or socket in the bone in FIG. 6A. In FIG. 6B, the Introducer 26 is placed in the hole, and in so doing, the tendon 25 is placed in the hole; and the interference screw 27 is inserted into the hole compressing the tendon 25 against the implant 18.

In FIG. 6D a variant of the procedure is shown where a rectangular strip implant 17 is placed on one side of the hole. Those skilled in the art recognize that there are many variations of surgical technique or fixation method that could be used in this instance and that implant shapes based on, or derived from, a rectangular prism could be utilized. The implant 17, 18, 20, 22, or 23 (FIGS. 5A-5E) may be placed prior to, or at the same time as, or after, insertion of the ligament or tendon.

In other embodiment, the rectangular strip implant 17 is placed in femoral socket 12 of the femur (first bone) and tibial tunnel 16′ in the tibia (second bone) for an acl reconstruction as is shown in FIG. 7A. The implant devices 17 including interference screw 41 may be placed prior to, or at the same time as, or after the graft 14 is placed into the socket 12 and graft tunnel 16′. While the fixation in this example is suspensory fixation 15 for the femur 40 (first bone) and an interference screw 41 for the tibia 60 (second bone), the implant placement would also be compatible with other fixation methods such as a cross-pin, cortical bone screws, and/or staples. With reference to FIGS. 7B and 7C, a cross-pin (or cortical bone screw) 54 is used to fix the sutures in place as shown in the tibial socket 45′ in FIG. 7B and in the femur socket 45 and tibial socket 45′ in FIG. 7C.

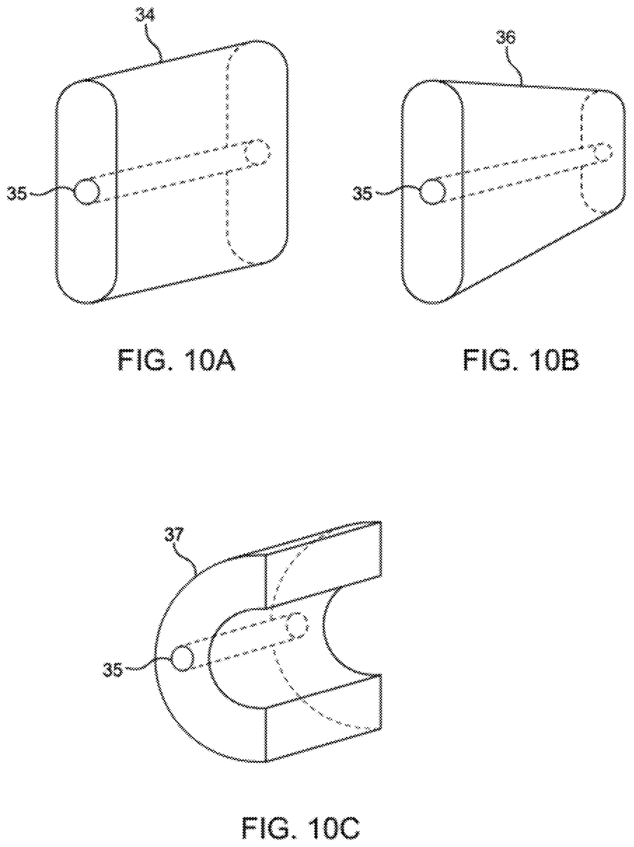

A rectangular prism implant 34 may be cannulated 35, as is shown in FIG. 10A. Other non-limiting variants of this implant include versions such as the tapered rectangular prism implant 36, or the cylindrical segment 37.

The implants (8, 10, 17, 18, 20, 22, 23, 34, 36, 37, 42, 43, 51, 53) according to embodiments of this disclosure, may be fabricated or formed from DBF. As such, the DBFs may be machined from cortical bone and then subsequently demineralized, or the DBFs may be machined from demineralized cortical bone.

In an exemplary embodiment, the implanting of implant 34 is shown in FIGS. 11A-11C. In this example, graft 38 is a 4-strand hamstring graft pulled into the bone (tunnel or socket) 39, as shown in FIG. 11A. As depicted, a guide wire 55 is placed in the (tunnel or socket) 39 as is shown in FIG. 11B. The implant 34 is then pushed down the guide wire 55 into the (tunnel or socket) 39. In doing this, the graft 38 is compressed, serving to improve its fixation. In some embodiments, the implant 34 is pushed down the wire using a knot pusher or a cannulated rod or similar. Implant 34 is shown in FIG. 11C, however, any of the cannulated implants 34, 36, 37, 51, 53 disclosed herein may be inserted into a (tunnel or socket) 39 using guide wire 55.

It is noted that the guide wire 55 is an exemplary method, but is not required. Alternatively, the fiber implants (8, 43) having a central hole (9 or 44) as disclosed herein, may be inserted into a (tunnel or socket) 39 using an introducer 33. Furthermore, any non-cannulated implant as disclosed herein (e.g., 17, 18, 20, 22, 23, 42) may also be posited by hand or using any suitable instrument into a (tunnel or socket) 39 to improve fixation of the graft 38. As depicted in FIGS. 11A-11C, and 12A-12B, the (tunnel or socket) 39 depicts a tunnel or a socket as defined herein. the proximal end through which the graft and implant are positioned using the introducer is open and the distal end may be closed like the socket 45, open from end to end like tunnel 16, 16′, or have a hole, as disclosed in other embodiments throughout this disclosure.

In additional embodiments, for revision surgery, the guide wire 55 may be placed to allow the implant 34 to push the graft to one side of the tunnel to allow the surgeon to correct for a tunnel that has been drilled in the wrong location. In revision surgery, multiple implants (e.g., the implants disclosed herein) may be placed to compensate for an enlarged tunnel. This may have the benefit of allowing a revision surgery to be completed in a single operation rather than having to apply bone graft in a first surgery and then reconstruct the ligament in a second surgery.

The rectangular prism implant (34, 36, 37) has a width of about 3 to about 8 mm, a height of about 3 to about 10 mm, and a length of about 10 to 100 mm. The cannulation is about 0.5 to about 4 mm. In some embodiments, the rectangular prism implant device has a width of 3, 4, 5, 6, 7, or 8 mm; a height of 3, 4, 5, 6, 7, 8, 9, or 10 mm; and a length of 10, 15, 20, 25, 30, 35, 40, 45, 50, 55, 60, 65, 70, 80, 85, 90, 95, or 100 mm. In specific embodiments, the length of the rectangular prism is about 10 to about 100 mm, about 10 to about 80 mm, about 10 to about 70 mm, about 10 to about 60 mm, about 10 to about 50 mm, about 20 to about 80 mm, about 20 to about 70 mm, about 20 to about 60 mm, or about 20 to about 50 mm. As would be understood by the skilled person, in some embodiments, where the width and the height of the rectangular prism approximate each other, the rectangle shape is or approximates a square.

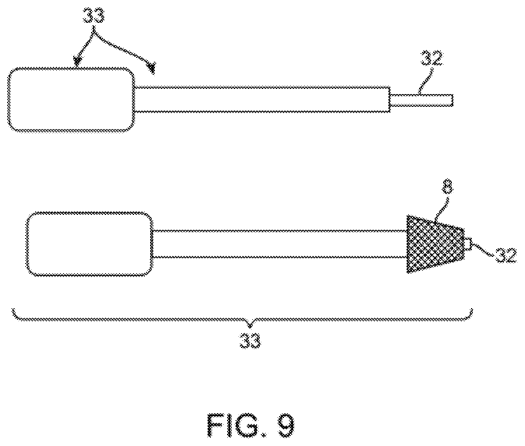

As an alternative to using a guide wire for a cannulated implant an insertion device 33 as shown in FIG. 9 may be used. The diameter of the distal feature 32 may be designed to provide an interference fit with the cannulation diameter of the implant. Alternatively, a short region 0.5 to 3 mm in length may be of an increased diameter to provide the interference. Cannulation of the device and the use of an insertion tool 33 greatly improves the ease of use for the surgeon and provides improved control over the placement of the implant.

A cannulated implant (e.g., 34, 36, 37, 51, 53) may also be incorporated into a hamstring or other graft at the time that it is being whipstitched in preparation for insertion into the patient. Suture may be threaded through the cannulation and the implant then whipstitched into the region of the graft that will be in the bone tunnels.

Interference screw fixation is a conventional and common means of graft fixation. It is also possible to adapt an interference screw 41 to allow it to facilitate delivery of a DBF implant. Interference screws are cannulated and allow insertion of a driver into the screw for screw insertion. To enable communication between the center of the screw and the outside the screw needs to be fenestrated either by inclusion of a number of drilled holes, as shown in FIG. 12, or by utilizing a suitable technique (e.g., 3D printing) to provide a screw with an open structure. Once the screw has been inserted and the driver and guide wire removed the space that these occupied can be replaced by an implant. This arrangement is shown in FIG. 12 where a multi-edge rod implant (e.g., hexagonal implant 42) is placed in the interference screw 41.

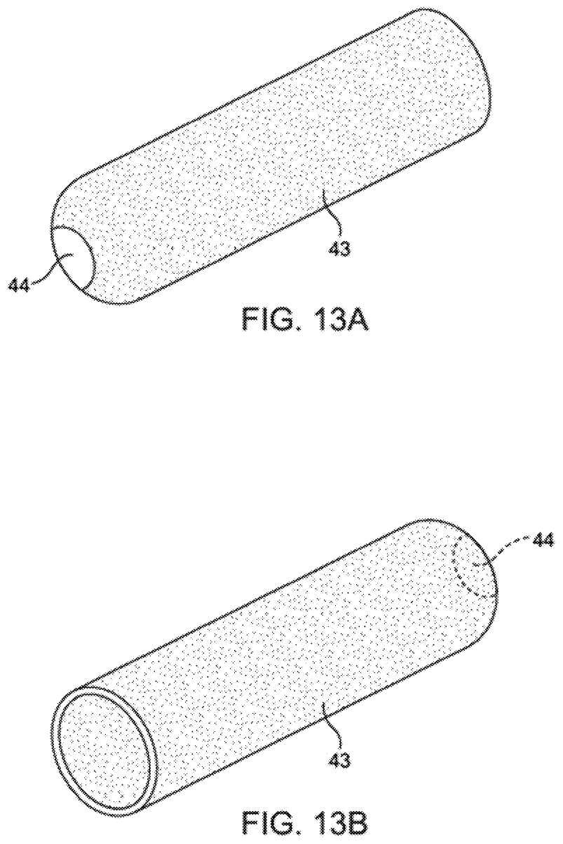

In another variant of the current invention shown in FIGS. 13A-13B the tubular implant 43 is in the form of a tube or bag that is placed in the bone tunnel either prior to introduction of the acl or tendon graft or with the acl or tendon graft. In some embodiments, the tubular implant 43 has a length of about 10 mm to about 40 mm, a diameter of about 5 to 10 mm, and a wall thickness of about 0.5 to 2 mm. In some embodiments, the length of the tubular implant 43 is about 10 mm to about 40 mm, about 10 to about 35 mm, about 10 to about 35 mm, about 10 to about 30 mm, about 10 to about 25 mm, or about 10 to about 20 mm. One end of the cylinder has an opening or hole 44 in it. The opening 44 is about 0.5 mm to 5 mm in diameter. The opening 44 allows for the threading through of the suture and/or button fixation devices as disclosed herein.

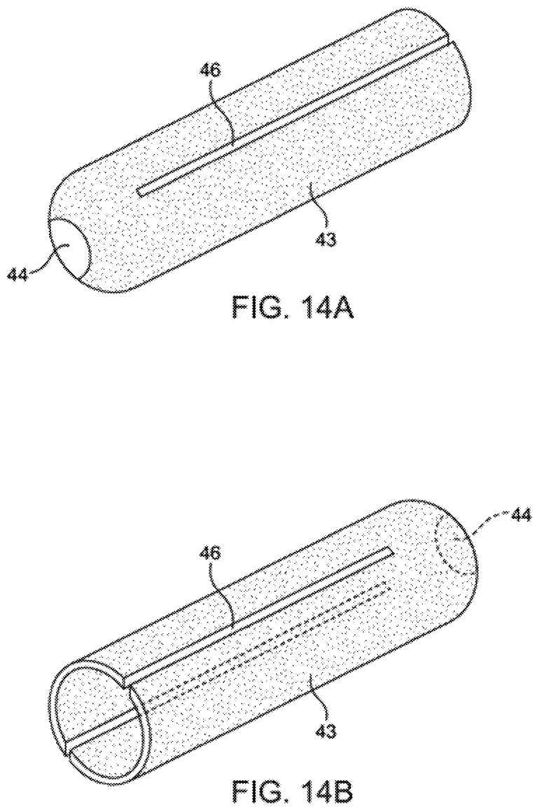

In another variant of the current invention shown in FIGS. 14A-14B, the implant 43 is in the form of a tube or bag that is placed in the bone tunnel either prior to introduction of the acl or tendon graft or with the acl or tendon graft. The tubular implant 43 has slits 46 along part of its length. The slits 46 may be between about 0.5 to 2 mm wide and there may be one or more slits in the implant. The one or more slits along part of the implant length may be about 10 mm to about 35 mm, so long as about 5 mm to about 15 mm of the implant does not comprise a slit to maintain the integrity of the implant.

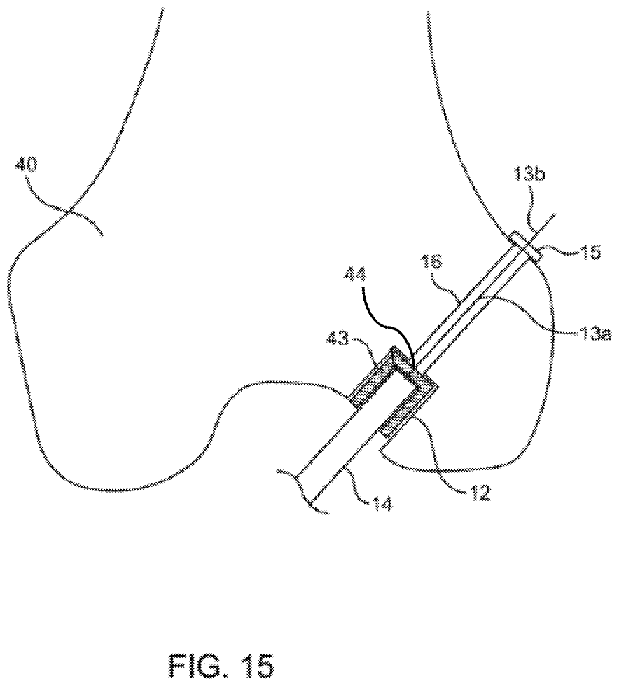

With reference to FIG. 15, the tubular implant 43 is placed in contact with the bone in femur socket 12 in a femur 40 for fixation of ligament or tendon graft 14 with sutures 13a, 13b attached to the tendon graft 14 and the suture(s) 13a, 13b extending through hole 44 in the implant and a hole at the distal end (opposite the opening) of the femoral socket 12 through the femoral tunnel 16 to the surface of the femur 40 for placement of a fixation device 15 (e.g., a button fixation device) thereon.

In order to prepare an acl graft for implantation outside of the patient a graft preparation board may be used to facilitate the process. The strands of the graft 14 are whipstitched to a suture 13a and fixation button 15 and pulled into the tubular implant 43. The graft construct may be compressed and further whipstitched to reduce its diameter to compensate for the additional bulk of the implant.

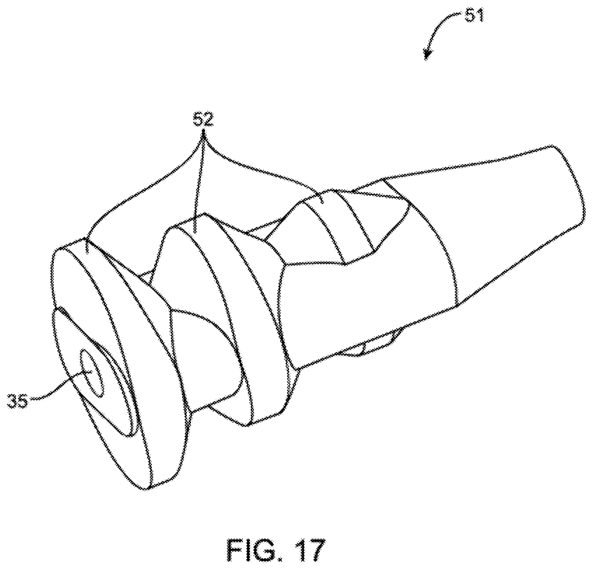

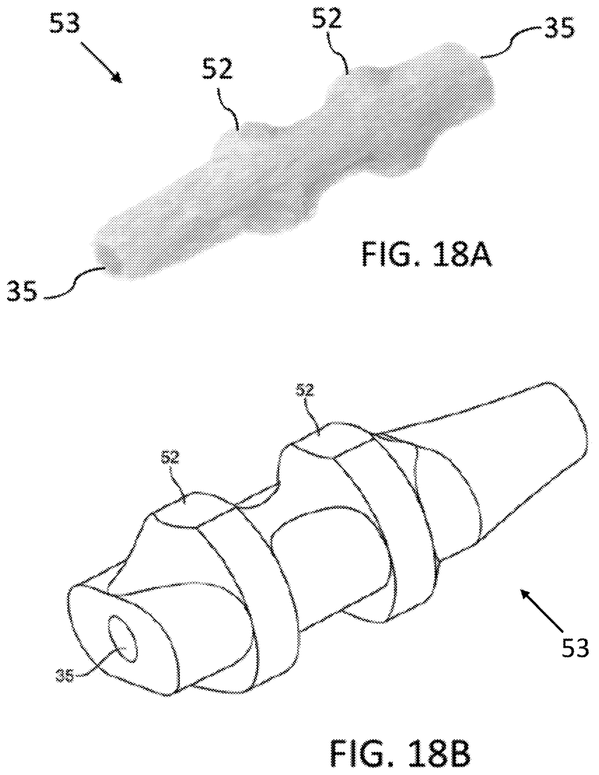

The rectangular prism implants 34, 36, 37 of FIGS. 10A-10C and 11A-11C may be further optimized by the addition of protuberances along their length. These protuberances 52 are designed to resist migration of the implant during ambulation. Examples of these are shown in FIG. 17 and FIGS. 18A-18B.

With reference to FIG. 17, a schematic of tubular prism implant 51 is depicted having a width of about 3 to about 8 mm, a height of about 3 to about 10 mm, and a length of about 10 to 100 mm, having three protuberances 52 along its length. The implant 51 has cannulation 35 having a diameter of about 0.5 to 4 mm. In some embodiments, the tubular prism implant device has a width of 3, 4, 5, 6, 7, or 8 mm; a height of 3, 4, 5, 6, 7, 8, 9, or 10 mm; and a length of 10, 15, 20, 25, 30, 35, 40, 45, 50, 55, 60, 65, 70, 80, 85, 90, 95, or 100 mm. In specific embodiments, the length of the rectangular prism is about 10 to about 100 mm, about 10 to about 80 mm, about 10 to about 70 mm, about 10 to about 60 mm, about 10 to about 50 mm, about 20 to about 80 mm, about 20 to about 70 mm, about 20 to about 60 mm, or about 20 to about 50 mm. The protuberances extend about 1 to 5 mm beyond the body of the implant. In some embodiments, as shown, the tubular prism 51 is tapered at each end.

With reference to FIG. 18A, a tubular prism implant 53 is shown that is made from DBF, having a width of about 3 to about 8 mm, a height of about 3 to about 10 mm, and a length of about 10 to 100 mm, and has two protuberances 52 along its length. The cannulation 35 has a diameter of about 0.5 to 4 mm. In some embodiments, the tubular prism implant device 53 has a width of 3, 4, 5, 6, 7, or 8 mm; a height of 3, 4, 5, 6, 7, 8, 9, or 10 mm; and a length of a length of 10, 15, 20, 25, 30, 35, 40, 45, 50, 55, 60, 65, 70, 80, 85, 90, 95, or 100 mm. In specific embodiments, the length of the rectangular prism is about 10 to about 100 mm, about 10 to about 80 mm, about 10 to about 70 mm, about 10 to about 60 mm, about 10 to about 50 mm, about 20 to about 80 mm, about 20 to about 70 mm, about 20 to about 60 mm, or about 20 to about 50 mm. The protuberances 52 extend about 1 to 5 mm beyond the body of the implant. FIG. 18B is a schematic depicting tubular prism implant 53 having a tubular prism shape and tapered ends.

Whereas the implants disclosed herein are shown in the context of acl repair in a femur, the disclosed implants (8, 10, 17, 18, 20, 22, 23, 34, 36, 37, 42, 43, 51, 53) allow enhancement of fixation for any ligament or tendon in any bone of the body including, without limitation, the posterior cruciate ligament, biceps tendon, anterior tibialis tendon, posterior tibialis tendon, quadriceps tendon, and flexor hallucis longus tendon. Grafts may be autologous, allogeneic, or xenogeneic. Accordingly, fixation using the presently disclosed implants may be in bones including, without limitation, the femur, tibia, foot, and humerus.

In still other embodiments, the disclosed implants (8, 10, 17, 18, 20, 22, 23, 34, 36, 37, 42, 43, 51, 53) are used in tendon transfer procedures.

In some embodiments of the present invention, the DBF used in an implant uses bone that has had the mineral component removed by a demineralization process that renders the graft malleable and not hard. The bone is then further formed into fibers by cutting along the long axis such that the collagen fibers within it are maintained in their natural fibrous form, as disclosed in U.S. Pat. Nos. 9,486,557 and 9,572,912, supra.

Suitable methods for forming implants from DBF are disclosed in WO 2016/123583, US 2018/0263799, and US 2020/0397586, the entire contents of all of which are herein incorporated by reference.

In some embodiments, the methods for making the bone fibers include demineralizing whole bone and subsequently cutting the demineralized bone in a direction parallel to the orientation of collagen fibers within the demineralized bone to form elongated bone fibers. The bone material of the present invention is derived from human (allograft) or animal (xenograft) cortical bone and is processed in such a manner to provide grafts of high utility based on the controlled geometry of the bone fibers. For veterinary applications bone from the same species. e.g., canine for canine patients (allograft) may be used as well as bone from other species (xenograft). It will be obvious to one skilled in the art that fibers other than demineralized bone fibers may be utilized to make a bone graft of this invention. Such fibers may be made from resorbable polymers or bioactive glasses or mixtures thereof, and may be used in place of or as an additive to the demineralized bone fibers (DBF). The methods of preparation of the graft provide improved efficiency and uniformity with reproducible results and decreased requirements for equipment and resulting costs. The implant device forms according to some embodiments of the present invention do not require the addition of exogenous materials to maintain the form of the graft. These improved characteristics will be apparent to one skilled in the art based upon the present disclosure.

The fibers need to have greater than a minimum length to be able to function effectively. If they are too short, they will not entangle to form a cohesive dry implant. The minimum length is between 10 and 20 mm, or approximately 15 mm. Fibers in the range of 0.15 cm to 4 cm in length have sufficient length to provide entanglement and are preferably 500 to 1500 microns in width and 50 to 300 microns thick. However, fibers having a length of 5, 6, 7, 8, 9, or 10 cm may also be combined with fibers having a length of 0.15 cm to 4 cm.

It is also important to a number of the applications that the DBF can be dried to render a stiffer implant at the time of implantation.

A further benefit of the DBF fibers is their ability to be processed to form an implant that retains its integrity when wet, such that it is suitable to be used in arthroscopic surgery.

Cortical bone could also be machined to fabricate implants of the strip designs shown in FIGS. 5A-5E, either prior to, or subsequent to, demineralization. Demineralized implants can then be utilized in the same manner as the implants fabricated from DBF.

### Processing of Fibers

Processing of the demineralized bone fibers, synthetic polymer fibers, collagen fibers, or resorbable polymer fibers to produce a desired shape or form of the fibers may be performed using any suitable method. Processing of specifically demineralized bone fibers may be performed using any suitable method as disclosed herein. To make some of these forms, the bone fibers may be collected, ideally in their hydrated state, and compressed using pressure molds, the pressure being sufficient to form the required shape but not so high as to lose the porosity of the fibrous structure. In some embodiments, the bone fibers are formed using a wet lay technique as is well understood by those skilled in the art of nonwoven or paper manufacture. Using a wet lay technique, the cut bone fibers are suspended in an aqueous solution to form a bone fiber slurry. Any suitable biocompatible aqueous solution may be used. Non-limiting examples of biocompatible aqueous solutions include: water, saline, and/or solutions including salts such as phosphate buffered saline (PBS), Ringer's solution, Lactated Ringer's solution, and saline with 5% dextrose. In some embodiments of the present invention, cut fibers are placed into saline to create a slurry of entangled bone fibers. The bone fiber slurry is suspended over a mesh screen (having holes) and the saline is drained resulting in a wet lay process, such that a sheet of demineralized bone fibers is formed on the mesh screen. The screen is contoured to provide a three-dimensional shape to the screen such that cylindrical pellets may be directly produced, or is flat so that a sheet is produced. The resulting devices may be then dried using heat and/or vacuum or other means such as lyophilization (freeze-drying). In some embodiments, prior to drying, the sheet is placed in a mold and compressed to a defined thickness and shape, followed by drying. As discussed herein, density, porosity and overall dimensions of the resulting product may be controlled using various molds and techniques.

Hydrated fibers may also be simply placed into a cylindrical mold cavity and lightly compressed using a plunger or push rod. A set amount of fiber is introduced into a cylindrical mold and the plunger used to compress the fibers to the required density through control of the depth that the plunger is pushed. Where a plunger has a spike on it, the spike may be designed to form a depression, a partial hole, or a hole through the length of the implant. In this latter instance the implant will be in the form of a tube.

In some embodiments a vacuum oven is used, whereby the application of vacuum removes moisture and dries the implant.

In some embodiments the heating step is undertaken by placing the implant in contact with a metal or other high heat-conductivity surface such that the degree of annealing/crosslinking is enhanced at that surface.

In other embodiments, the bone fibers are further processed in a second drying step that may include vacuum drying and/or lyophilization.

In some embodiments the amount of compression, heating, and drying can be tailored to modify the rehydration and re-expansion rates. For example, with no heating the rehydration is very fast whereas heating at or between 35° to 55° or 45° to 55° C. for approximately one hour causes very slow re-hydration and re-expansion. In other aspects, the heating may occur at any of 35°, 36°, 37°, 38°, 39°, 40°, 41°, 42°, 43°, 44°, 45°, 46°, 47°, 48°, 49°, 50°, 51°, 52°, 53°, 54°, or 55° C. By altering these processes, bone fiber compositions as disclosed herein may retain their manufactured shape during packaging, shipment, unpacking and placement into the graft site, but after placement into the graft site the DBF will begin to absorb moisture rapidly (within 30 seconds or less) and may be completely re-hydrated/re-expanded within approximately 2 minutes, preferably being completely re-hydrated/re-expanded within 30 seconds.

In other embodiments the bone fibers may retain some moisture and will be placed in moisture impervious packaging. Furthermore, the dried and molded bone fiber implant may be sterilized after packaging. Sterilization of the implant may be carried out using any suitable method. For example, sterilization may be carried out by electron beam or gamma beam. Alternatively, aseptic manufacture may be used to avoid the need for terminal sterilization.

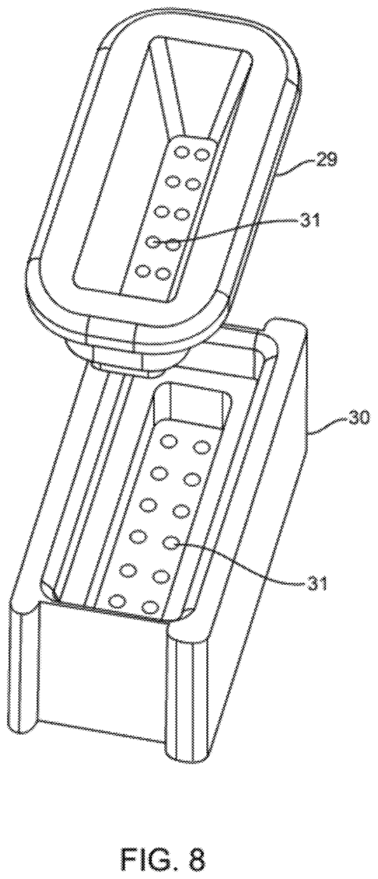

In other embodiments, the inventive subject matter also includes a simple mold of the sort shown in FIG. 8 may be used to make DBF sheets of 0.5 mm to 5 mm thick, where the mold lid 29 may be placed on the mold 30 (the mold having holes 31 for drainage of the liquid in the DBF slurry) where the lid is in contact with the DBF after the DBF has been wet laid and may define the degree of compression of the DBF and hence the density of the sheet. Protrusions on the lid or base may be used to form the compressed areas, e.g. 19 in FIG. 5B (protrusions not shown).

A DBF sheet that is dried will have a low wet strength when rehydrated and improvement to the DBF sheet wet strength may be affected by placing the mold in an oven at 45-55° C. and heat treating the sheet for up to 2 hours.

In some embodiments, cylindrical or conical implants such as those shown in FIGS. 2 and 3 are formed by adding wet fibers directly into a cylindrical or conical mold. The mold is loaded with the fiber. A tamp is used to apply some compression to the fibers. In some embodiments, a fiber loaded cylindrical mold is dried by heat, vacuum, and/or lyophilization. After drying, the bone fiber implant becomes more cohesive and shrinks to a reduced volume. After drying, the bone fiber pellets may be easily expelled out of the mold due to the shrinkage that occurs upon drying.

While wet lay techniques may be used for the manufacture of different shapes from the bone fibers, it will be recognized that any other molding or forming technique used with textile fibers could be used. Fibers with and without excipients may be directly molded using compression into any shape. In some embodiments, excipients may be selected that enhance the lubricity of the implant facilitating delivery and further reducing and friction or binding during this procedure.

The wet lay process was originally developed for use in paper making and textiles where the fibers are processed to make a two-dimensional sheet-like product. As the fluid drains the fibers are laid onto the surface of the mold and as such are in a plane that is generally parallel to the plane of the sheet being produced. While this process can accommodate some undulations and be used to make shapes like egg cartons it is wholly unsuitable for the fabrication of cylindrical shapes. The size of the mold may be large enough to allow multiple implants to be cut from the formed sheet or it may be sized to make an individual implant.

A water assisted injection molding (WAIM) process has been developed to allow the manufacture of non-sheet like implants. The required mass of DBF to fill molds for the water assisted injection molding process is approximately 0.15 gram to 1 gram and may be dispersed in about 20 mls of fluid in a syringe, in a ratio of fibers to fluid as disclosed herein. Dispersion of the fibers in fluid into the molds may be by injection pressure or by vacuum. Any suitable fluid buffer may be used. For example, phosphate buffered saline (PBS) may be used for dispersion of the fibers as well as water or any biocompatible buffer or liquid.

In contrast to conventional methods of fabrication, rather than rely on gravitational flow, an elevated pressure is applied to the dilute fiber and fluid dispersion. This forces the fibers to flow down narrow diameter structures rather than form an entangled clump at the entrance to the mold cavity. Because the fibers are dispersed, they do not compact when introduced into the mold. Accordingly, the slurry suspension of fibers is advantageously introduced and place in the mold because the fiber suspension is injected or pushed into the mold at a rate greater than a gravitational flow, but not so much pressure/force that the fibers clump or compact. For example, a 15 ml slurry of a fiber suspension having a ratio as disclosed herein is introduced into a mold in about 1 to 5 seconds. As such, the slurry of fibers is placed or provided into the mold at a rate from about 3 ml/second up to 15 ml/second.

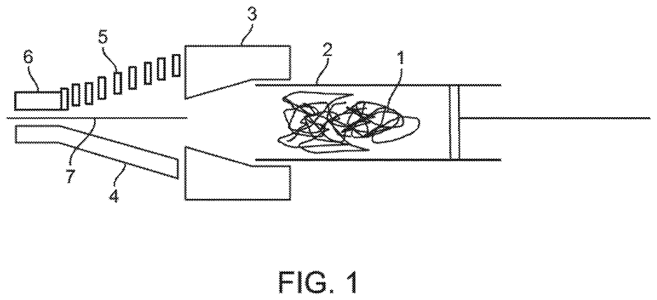

FIG. 1 depicts an apparatus for water-or fluid-assisted injection molding of DBF fibers. The required mass of DBF fibers 1 is loaded into a syringe 2. A suitable fluid (e.g., water, saline, or a buffered saline including phosphate buffered saline (PBS) is then added to the syringe. The distal end of the syringe is then fitted into an adapter 3 to which a detachable mold 4 is attached. The mold renders the required dimensions of the implant to be made, and the mold may be cylindrical, ribbed, and/or tapered. As with conventional injection molding, the cylinder will have a small taper or draft to allow removal of the molded part. The mold is tapered towards its distal end and has vents 5 along its length, and a removable vented end cap 6 to allow for the fluid to egress out of the DBF mixture. When a cannulated implant is desired a cannulating wire 7 is inserted into the mold prior to, or after, molding. The detachable mold is removed after DBF injection and placed into an oven or lyophilizer for drying. Multiple molds may be used with one adapter and syringe to allow multiple parts to be fabricated. Importantly, the porosity of the demineralized bone fibers (DBFs) is maintained when forming the DBF fiber implants disclosed herein. Accordingly, a suitable amount of compression pressure when applied to the mold does not result in a loss of porosity and fibrous structure of the DBF implant.

In some embodiments of the present invention, the presently disclosed implant is made with a ratio of fluid (e.g., saline) to DBF of about 3 mls fluid to 1 gram DBF. In other embodiments, the ratio of fluid to DBF is about 3, 4, 5, 6, 7, 8, 9, 10, 11, 12, 13, 14, 15, 16, 17, 18, 19, 20, 21, 22, 23, 24, 25, 26, 27, 28, 29, 30, 40, 41, 42, 43, 44, 45, 46, 47, 48, 49, 50, 51, 52, 53, 54, 55, 56, 57, 58, 59, or 60 mls fluid to 1 gram of DBF. In still other embodiments, the ratio of fluid to DBF is less than about 200 mls fluid to 1 gram DBF. Advantageously, a ratio of fluid to DBF being in the range of between about 3 to 200 mls fluid to 1 gram DBF, 3 to 150 mls to 1 gram DBF, 3 to 100 mls to 1 gram DBF, 3 to 50 mls to 1 gram DBF, 3 to 20 mls to 1 gram DBF, or 3 to 10 mls to 1 gram DBF all provide enough fluid to hydrate the DBF to facilitate shaping and forming of the DBF implant into the desired shape.

In similar embodiments, the weight amount of DBF in the presently disclosed is about 0.25 grams up to about 1.0 gram. Accordingly, the weight amount of DBF in an implant composition may be 0.25, 0.30, 0.40, 0.45, 0.50, 0.55, 0.60, 0.65, 0.70, 0.75, 0.80, 0.85, 0.90, 0.92, 0.95, 0.96, 0.97, 0.98, 0.99, or 1.0 gram. Accordingly, 0.25 to 1 gram DBF is dispersed in 20 to 50 mls of fluid.

Subsequent to molding using the WAIM process implants in their molds may be heated to improve cohesiveness when wet. The molds may be placed into a convection oven at 35-55° C. for 60 to 180 minutes. In other aspects, the heating may occur at any of 35°, 36°, 37°, 38°, 39°, 40°, 41°, 42°, 43°, 44°, 45°, 46°, 47°, 48°, 49°, 50°, 51°, 52°, 53°, 54°, or 55° C. Following the heat treatment the molds may then be lyophilized or vacuum dried to remove any residual moisture.

The implants of the present disclosure in their dry state may be inserted into a cavity, awl hole, or drill hole. Additionally, the implants of the present disclosure may be housed in a syringe or syringe-like insertion device. With the implant in a syringe or syringe-like insertion device, the implant may have lateral stability thereby preventing or decreasing bending or buckling of the implant while it is being pushed into the surgical site (e.g., the cavity or hole).

In some embodiments, a delivery instrument 33 depicted in FIG. 9 is used to provide the implant into the bone site to be repaired. The cannulation of the implant 8 is designed to be an interference fit on the proximal portion 32 of the delivery instrument 33.

Alternatively, any of the cannulated implants (34, 36, 37, 51, 53) may be introduced into the surgical site using a guide wire 55.

### Excipients and Additives

Additives are contemplated to modify biological or other properties of the implant according to embodiments of the present invention. Non-limiting examples of additives include growth factors such as bone morphogenetic proteins (BMPs), including BMP-1, BMP-2, BMP-3, BMP-4, BMP-5, BMP-6, BMP-7, BMP-8, BMP-9, BMP-10, BMP-11, BMP-12, BMP-13, BMP-15, BMP-16, BMP-17, and BMP-18; Vascular Endothelial Growth Factors (VEGFs), including VEGF-A, VEGF-B, VEGF-C, VEGF-D and VEGF-E; Connective Tissue Growth Factors (CTGFs), including CTGF-1, CTGF-2, and CTGF-3; Osteoprotegerin, Transforming Growth Factor betas (TGF-βs), including TGF-β-1, TGF-β-2, and TGF-β-3, and inhibitors for tumor necrosis factor (e.g., anti-TNF-α). Morphogens may also include Platelet Derived Growth Factors (PDGFs), including PDGF-A, PDGF-B, PDGF-C, PDGF-D, and GDF-5; rhGDF-5; and LIM mineralization protein, insulin-related growth factor-I (IGF-I), insulin-related growth factor-II (IGF-II), fibroblast growth factor (FGF) and beta-2-microglobulin (BDGF II), as disclosed in the U.S. Pat. No. 6,630,153, the entire contents of which is incorporated herein by reference. The polynucleotides encoding the same may also be administered as gene therapy agents. The preferred bioactive substances are the recombinant human bone morphogenetic proteins (rhBMPs) because they are available in relatively unlimited supply and do not transmit infectious diseases. In some embodiments, the bone morphogenetic protein is a rhBMP-2, rhBMP-4, rhBMP-7, or heterodimers thereof. BMPs are available from Wyeth, Madison, N.J., and may also be prepared by one skilled in the art as described in U.S. Pat. No. 5,366,875 to Wozney et al.; U.S. Pat. No. 4,877,864 to Wang et al.; U.S. Pat. No. 5,108,922 to Wang et al.; U.S. Pat. No. 5,116,738 to Wang et al.; U.S. Pat. No. 5,013,649 to Wang et al.; U.S. Pat. No. 5,106,748 to Wozney et al.; and PCT Patent Nos. WO 93/00432 to Wozney et al.; WO 94/26893 to Celeste et al.; and WO94/26892 to Celeste et al., the entire contents of all of which are herein incorporated by reference.

Oxygenating additives such as perfluorocarbons may be used to further enhance the bone formation and healing of the DBF material in the implant of the present disclosure. In some embodiments, the bone repair DBF implant composition includes oxygenating materials such as a perfluorocarbon (PFC). In some embodiments, the DBF implant composition includes oxygen generating compounds such as peroxides (e.g., hydrogen peroxide, magnesium peroxide, calcium peroxide), perchlorates (e.g., sodium perchlorate, potassium perchlorate), percarbonates (e.g., sodium percarbonate), or perborates (e.g., sodium perborate).

For additional benefits, cancellous or cortical bone chips and/or demineralized cancellous or cortical bone chips may be added to the DBF. In addition to or alternatively to the bone chips, mineralized bone fibers may be added to the DBF. In addition to bone chips, mineralized bone fibers or alternatively, calcium phosphate, tri-calcium phosphate, hydroxyapatite, or other synthetic bone graft materials may be added to the DBF.

At the time of surgery and prior to implantation, a small amount of sterile water, phosphate buffered saline, bone marrow aspirate, platelet rich plasma (PRP), and/or blood may be injected into the implant to hydrate the implant.

In other aspects, embodiments of the present invention include DBF, collagen, polymer, and/or resorbable polymer fiber implants, and methods forming DBF, collagen, polymer, and/or resorbable polymer fibers implants, and kits including suitably formed DBF, collagen, polymer, and/or resorbable polymer fiber implants for use as an interface between the bone and the ligament or tendon to be repaired.

In some embodiments of the present invention, an implant system package or implant kit includes the introducer 33 and the conical and cylindrical implants (8, 10) as shown in FIG. 9.

In some embodiments of the present invention, an implant system package or implant kit includes one or more of the suitably shaped fiber implants as disclosed herein, and a guide wire. In additional embodiments, the kit includes one or more fiber implants and a fixation device (e.g., a suspensory fixation device), and/or a guide wire.

The implant may be supplied as a kit with a guide wire, introducer, and/or pusher as disclosed herein. The kit may also include a fixation device (e.g., a button fixation device and/or a cross-pin and/or screws. The guide wire and pusher may be supplied as sterile disposable single use items or may be designed to be re-usable. The inventors have also contemplated a kit for augmenting fixation of a ligament or tendon, wherein the kit includes at least one of the presently disclosed fiber implants as disclosed above and herein. The contemplated kit may also include a guide wire, a pusher, a drill, an awl or a tap, and/or a screw to be place in the bone to be repaired. In some embodiments, the guide wire, the awl, and/or the tap are disposable.

As used herein, the term “about” refers to a reasonable range about a value as determined by the practitioner of skill. The term “about” is used interchangeably with “approximately”. In certain embodiments, the term about refers to ±one, two, or three standard deviations. In certain embodiments, the term about refers to ±1%, 2%, 3%, 4%, 5%, 6%, 7%, 8%, 9%, 10%, 11%, 12%, 13%, 14%, or 15% of the relevant metric unit, for example, an amount, a length, width, thickness, or temperature (degrees). For example, about 5 mm includes 5.2 mm, which is 4% more than 5 mm.

### EXAMPLES

The following examples use cortical human bone. As discussed herein, either human or animal bone may be used as a source of cortical bone. Fibers were produced using the methodology as described in U.S. Pat. Nos. 9,486,557 and 9,572,912, supra. DBF fiber weights referred to below relate to hydrated DBF.

### Example 1

A 7 mm diameter mold, 10 mm long, with a 3 mm diameter cannulating rod was attached to a syringe into which 0.25 grams of DBF fiber was suspended in 15 mls of water. The syringe plunger was used to push the DBF slurry into the mold. The mold was detached from the syringe and a cap was placed on the mold to maintain the central positioning of the cannulating rod. The mold was placed into an oven at 55° C. for 90 minutes and then into a vacuum oven where it was dried overnight at room temperature. The implant was removed from the mold.

### Example 2

A mold 5 mm wide and 25 mm long was used to produce a strip like product. 0.25 grams of DBF was suspended in approximately 50 ml of water and introduced to the mold. After the water was drained from the mold a lid was fitted to the mold to compress the DBF. The mold was placed into an oven at 55° C. for 90 minutes and then into a vacuum oven where it was dried overnight at room temperature. The implant was removed from the mold.

### Example 3

15 grams of DBF fiber were wet laid using water in a 10 cm×11 cm flat mold to produce a sheet of DBF. The mold was heated at 50° C. for two hours to bond the fibers and dry the sheet. The sheet was approximately 1 mm thick. Strip implants 5 mm wide and 20 mm long were cut from the sheet.

### Example 4

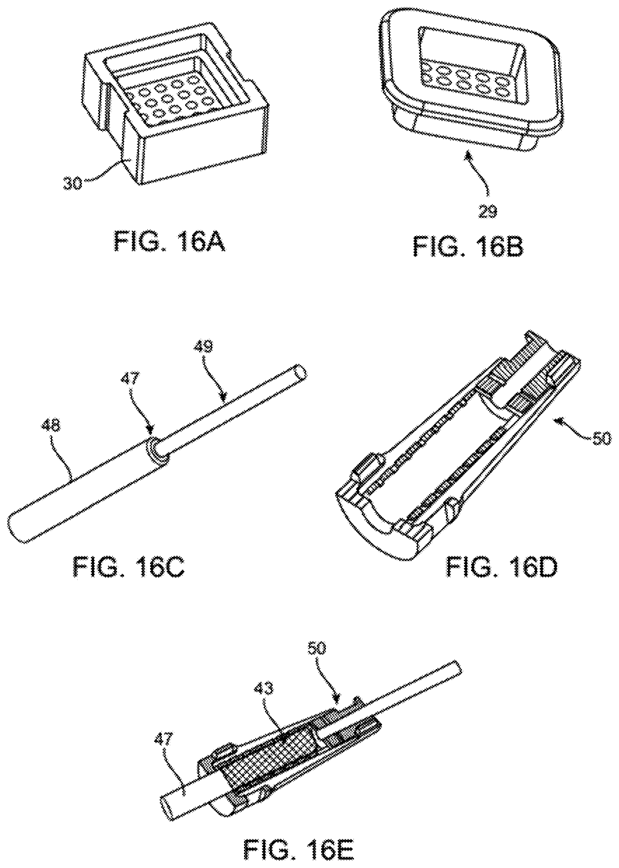

A cylindrical implant 43, as shown in FIGS. 14A-14B was produced using the apparatus as shown in FIGS. 16A-16E. Accordingly, 0.6 grams of DBF was wet laid using water in a 25 mm×50 mm flat mold (29, 30) to produce a sheet of DBF. The DBF sheet was removed from the mold 30 and placed on a piece of non-linting clean room fabric. With reference to FIG. 16C, the DBF sheet was wrapped around region 48 (6 mm diameter) of mandrel 47 and placed in a mold 50 with a 7 mm diameter to define the external diameter of the cylindrical implant. The two halves of the mold 50 (only one half shown in FIGS. 16D, 16E) were held in place using a clamp ring (not shown) and the mold was placed in an oven at 55° C. for 90 minutes. The mold 50 was then placed in a vacuum oven and the implant dried. The dried implant 43 was removed from the mold 50 and trimmed to remove the small amount of flash. The resultant implant was 25 mm long with a 6 mm internal diameter corresponding to region 48 and a 7 mm external diameter defined by the outer wall of mold 50. In some embodiments, cylindrical implants fabricated using this methodology have a slit 46 cut into them.

### Example 5

A mold 4 as shown in FIG. 1 was fabricated wherein the internal shape of the mold was designed to match the design of implant 53 shown in FIGS. 18A, 18B, having two protuberances 52. Accordingly, 0.52 grams of DBF was suspended in water in the syringe 2 of FIG. 1 used to inject the DBF into the mold 4. A cannulated cap end was placed on the mold 4 and a 1.5 mm diameter cannulating wire 7 was pushed through the mold to cannulate the implant. The two halves of the mold 4 were held in place using a clamp ring (not shown) and the mold placed in an oven at 55° C. for 90 minutes. The mold 4 was then placed in a vacuum oven and the implant dried. The dried implant 53 was removed from the mold 4 and trimmed to remove the small amount of flash. The resultant implant was approximately 17 mm long, 6.7 mm wide (maximum), and 5.5 mm thick (maximum).

While the present invention has been illustrated and described with reference to certain exemplary embodiments, those of ordinary skill in the art will understand that various modifications and changes may be made to the described embodiments without departing from the spirit and scope of the present invention, as defined in the following claims.

Additionally, although relative terms such as “outer,” “inner,” “upper,” “lower,” “below,” “above,” “vertical, “horizontal” and similar terms have been used herein to describe a spatial relationship of one element to another, it is understood that these terms are intended to encompass different orientations of the various elements and components of the device in addition to the orientation depicted in the figures.

What is claimed is:

1. An implant system for augmenting reattachment of a tendon or ligament to a bone, the implant system, comprising:
a tendon or ligament graft; and
a cannulated fiber implant, comprising a plurality of cut fibers comprising fibers selected from the group consisting of demineralized bone fibers (DBF), biocompatible polymer fibers, collagen fibers, resorbable polymer fibers, and a combination thereof,
wherein the plurality of cut fibers having a shape selected from a tubular prism with at least one protuberance, a rectangular prism with rounded ends, a tapered rectangular prism with rounded ends, or a cylindrical segment, and
wherein the cannulated fiber implant having one or more diameters of about 3 to about 15 mm and a length of about 10 to about 50 mm.

2. The implant system of claim 1, wherein the cannulated fiber implant is placed in a tunnel or socket of the bone in contact with a portion of the tendon or ligament graft.

3. The implant system of any one of claims 1-2, comprising more than one cannulated fiber implant.

4. The implant system of any one of claims 1-3, wherein the cut fibers comprise DBF.

5. The implant system of any one of claims 1-4, wherein the tendon or ligament is selected from anterior cruciate ligament (acl), posterior cruciate ligament, biceps tendon, anterior tibialis tendon, posterior tibialis tendon, and flexor hallucis longus tendon.

6. The implant system of any one of claims 1-5, wherein the shape is the tubular prism with at least one protuberance, wherein the tubular prism implant has a width of about 3 to about 8 mm, a height of about 3 to about 10 mm, and a length of about 20 to 50 mm, and the at least one protuberance extends about 1 to 5 mm outward from the body of the implant.

7. The implant system of claim 6, wherein the tubular prism comprises at least two protuberances.

8. The implant system of any one of claims 1-7, wherein the plurality of fibers comprise DBF and the cannulated fiber implant forms a tubular prism with tapered ends and at least one protuberance, the implant having a width of about 3 to about 8 mm, a height of about 3 to about 10 mm, and a length of about 20 to 50 mm, and the at least one protuberance extending about 1 to 5 mm outward from the implant.

9. The implant system of any one of claims 1-8, further comprising a fixation device.

10. A cannulated fiber implant for tendon or ligament graft fixation, the cannulated fiber implant comprising:
a plurality of cut fibers comprising fibers selected from the group consisting of demineralized bone fibers (DBF), biocompatible polymer fibers, collagen fibers, resorbable polymer fibers, and a combination thereof,
the plurality of cut fibers having a shape of a cannulated tubular prism with tapered ends and at least one protuberance, the implant having a width of about 3 to about 8 mm, a height of about 3 to about 10 mm, and a length of about 20 to 50 mm, and the at least one protuberance extending about 1 to 5 mm outward from the implant,
optionally wherein the cannulated fiber implant is formed from demineralized bone fiber (DBF);
optionally wherein the cannulated fiber implant comprises at least one protuberance;
optionally wherein the cannulated fiber implant has a width of about 3 to about 8 mm, a height of about 3 to about 10 mm, and a length of about 20 to 50 mm; and
optionally wherein the cannulation has a diameter of about 0.5 to about 4 mm; and has two protuberances along its length.

11. A method of augmenting reattachment of a tendon or ligament graft to a first bone using the implant system of any one of claims 1-9 or the cannulated fiber implant of claim 10.

12. A method of augmenting reattachment of a tendon or ligament graft to a bone of a subject in need thereof, using the implant system of any one of claims 1-9 or the cannulated fiber implant of claim 10, the method comprising:
positing the cannulated fiber implant in contact with the tendon or ligament graft in a tunnel and/or a socket formed in the bone of the subject.

13. The method of claim 12, wherein the cannulated fiber implant is posited between the tendon or ligament graft and at least a portion of the tunnel and/or the socket.

14. The method of any one of claims 12-13, wherein positing the cannulated fiber implant into the tunnel and/or socket comprises using a guide wire.

15. The method of any one of claims 12-14, wherein the method further comprises forming the tunnel and/or the socket in the bone.

16. The method of any one of claims 12-14, wherein the bone is selected from the tibia, the femur, the humerus, the ulna, or the radius.

17. The method of any one of claims 12-16, wherein the forming of the tunnel and/or the socket comprises a tunnel and/or a socket in a femur and an opposing tunnel and/or socket in a tibia of the subject.

18. The method of any one of claims 12-17, wherein tendon or ligament graft is attached to a suture and the suture is attached to a fixation device.

19. The method of claim 12-17, wherein the fixation device is a button fixation device placed on a surface of the bone or the fixation device is a cross-pin.

20. The method of any one of claims 11-19, wherein the tendon or ligament graft is for anterior cruciate ligament (ACL) repair.

21. A fiber strip implant for augmenting reattachment of a tendon or a ligament graft to a bone, the fiber strip implant comprising:
a plurality of cut fibers comprising fibers selected from the group consisting of demineralized bone fibers (DBF), biocompatible polymer fibers, collagen fibers, resorbable polymer fibers, and a combination thereof; and
the plurality of fibers molded to form the strip having a length in a range from about to about 50 mm, a width in a range from about 3 to about 8 mm, and a thickness in a range from about 1 to about 3 mm.

22. The fiber strip implant of claim 21, wherein the plurality of cut fibers comprises DBF.

23. A method of augmenting reattachment of a tendon or ligament to a bone, the method comprising:
positing the tendon or ligament in a tunnel or socket in the bone; and
positing at least one fiber strip implant of claim 21 or claim 22 into the tunnel or socket,
wherein the fiber strip implant is positioned in between and in contact with a portion of the tunnel or socket and the tendon or ligament.

24. The method of claim 23, wherein the bone is selected from the tibia, the femur, the humerus, the ulna, or the radius.

25. The method of any one of claims 23 or 24, further comprising:
positing an interference screw in the tunnel or socket, thereby compressing the tendon or ligament graft against the fiber strip implant.

26. The method of any one of claims 23-25, wherein the tendon or ligament is stitched to a suture and is connected to an introducer, and wherein the positing of the tendon or ligament comprises using the introducer.

27. The method of claim 26, wherein the tendon or ligament is whipstitched to the suture.

28. The method of any one of claims 23-27, wherein the fiber strip implant comprises a plurality of fibers of DBF.

29. An implant system for augmenting reattachment of a tendon or ligament graft to a tunnel or socket in a bone, the implant system, comprising:
the tendon or ligament graft;
a cannulated interference screw; and
a fiber implant, comprising a plurality of cut fibers comprising fibers selected from the group consisting of demineralized bone fibers (DBF), biocompatible polymer fibers, collagen fibers, resorbable polymer fibers, and a combination thereof, the fiber implant molded into a multi-edged rod shape having a diameter in a range from about 3 to about 15 mm and a length in a range from about 3 to about 15 mm,
wherein the interference screw and the tendon or ligament graft are independently posited in the cavity or the tunnel in the bone, and
wherein the fiber implant is posited in the cannulated interference screw.

30. The implant system of claim 29, wherein the plurality of cut fibers comprises DBF.

31. The implant system of any one of claims 29 or 30, wherein the multi-edge rod shape is hexagonal, thereby forming a fiber implant having edges corresponding to the cannulated interference screw.

32. A method of augmenting reattachment of a tendon or ligament graft in a tunnel or socket in a bone, the method comprising implanting the implant system of any one of claims 29-31.

33. An implant system for augmenting reattachment of a tendon or ligament graft to a bone, the implant system, comprising:
the tendon or ligament graft;
a fixation device attached to an end of the tendon or ligament graft with a suture; and
the fiber implant comprising a plurality of cut fibers comprising fibers selected from the group consisting of demineralized bone fibers (DBF), biocompatible polymer fibers, collagen fibers, resorbable polymer fibers, and a combination thereof,
wherein the plurality of cut fibers is molded into a shape selected from a tube or a truncated cone each having a center hole therein,
wherein the fiber implant comprises one or more diameters of about 3 to about 15 mm and a length of about 10 to about 40 mm; and
wherein the tendon or ligament graft with the attached fixation device is threaded through the central hole.

34. The implant system of claim 33, wherein the fiber implant further comprises a slit down the length of the implant parallel to the central hole.

35. The implant system of claim 33 or claim 34, wherein the cut fibers comprise DBF.

36. A fiber implant for augmenting reattachment of a tendon or ligament graft to a bone, the fiber implant, comprising:
a plurality of cut fibers comprising fibers selected from the group consisting of demineralized bone fibers (DBF), biocompatible polymer fibers, collagen fibers, resorbable polymer fibers, and a combination thereof;
wherein the plurality of cut fibers is molded into a shape selected from a tube or a truncated cone each having a center hole therein, and
wherein the fiber implant comprises one or more diameters of about 3 to about 15 mm and a length of about 10 to about 40 mm.

37. A method of augmenting reattachment of a tendon or ligament graft to a bone of a subject, using the implant system of any one of claims 33-35 or the fiber implant of claim 36, the method comprising:
threading the fixation device, the tendon or ligament graft, and the fiber implant into a tunnel or socket of the bone.

38. The method of claim 37, wherein the fixation device is a button fixation device or a cross-pin.

39. The method of any one of claims 37-38, wherein the fiber implant device is posited into the tunnel or socket using an introducer.

40. The method of any one of claims 37-39, wherein the plurality of cut fibers is DBF.
